(19) United States
(12) Patent Application Publication
(10) Pub. No.: US 2006/0063225 A1
(43) Pub. Date: Mar. 23, 2006

(54) Novel procedure for growth, imaging, and enumeration of microbial colonies for serological or screening assays

(76) Inventors: Michael J Caulfield, Fort Washington, PA (US); Xu Liu, North Wales, PA (US); Su Wang, Schwenksville, PA (US)

Correspondence Address: MERCK AND CO., INC, P O BOX 2000, RAHWAY, NJ, 07065-0907, US

(21) Appl. No.: 10/522,280

(22) PCT Filed: Jul. 30, 2003
(86) PCT No.: PCT/US03/23928

§ 371 Date: Jan. 24, 2005

Related U.S. Application Data
(60) Provisional application No. 60/400,332, filed on Aug. 1, 2002.

(51) Int. Cl. C12Q 1/04

(52) U.S. Cl. 435/34

(57) ABSTRACT

A novel method is provided, for use within serological or screening assays, wherein microbial colonies are grown on filter membranes in multi-well plates. This process enables the colonies to be stained, imaged, and counted automatically using such automated systems as, e.g., computer and video-based imaging systems.

### CROSS-REFERENCE TO RELATED APPLICATIONS

The present application claims priority to provisional application U.S. Ser. No. 60/400,332, filed Aug. 1, 2002, respectively, hereby incorporated by reference.

### BACKGROUND OF THE INVENTION

Microorganisms and the impact they exert on resident hosts are of great concern and importance and the continued study thereof commands great resources. Microorganisms, while indispensable components of our ecosystem and significant contributors to the production of various important consumer products such as antibiotics, vaccines, vitamins, and enzymes, also have very negatively impacted humans and disrupted society over the millennia. For this reason, one essential area of focus in the area of microbiology is in the evaluation of various treatment regimens (e.g., antibiotics) on the viability of various microorganisms; e.g., bacteria, viruses, algae, fungi, and protozoa.

Bacterial pathogens, for instance, are continuously evolving a more refined and increasingly powerful resistance towards proven-effective antibacterial drugs. This trend and the resulting drug- and multi-drug-resistant pathogens have become more of an issue in the last decade. As a result, efforts have largely focused on identifying alternatives to the current arsenal of available antibacterial drugs.

Vaccination through the elicitation of a host immune response offers a very effective alternative to administering antibacterial drugs and has remained the focus of ongoing antibacterial efforts. Antibacterial vaccines are being developed at a very rapid rate. More, assays useful in evaluating the efficacy of developed vaccines and various antimicrobial agents are continuously being scrutinized and further optimized. This latter focus of optimization of assays and, in a preferred embodiment, antibacterial assays is advanced herein. Accordingly, a brief discussion of two basic types of assays utilized in the evaluation of antibacterial agents follows.

The first type of assay detects specific antibodies generated in response to a bacterial antigen or an administered vaccine. Assays capable of functioning in this capacity include radioimmunoassays (RIAs) and enzyme-linked immunosorbent assays (ELISAs); see, e.g., Schiffman et al., 1980 J. Immunol. Methods 33:13344; Nahm et al., 1996 J. Infect. Dis. 173:113-118; Quataert et al., 1995 Clin. Diagn. Lab. Immunol. 2:590-597. Briefly, assays of this nature are directed at measuring total binding and do not provide information regarding functionality of the binding antibodies.

The proper functioning of specific antibodies in question is investigated by a second type of assay capable of measuring the efficacy of the antibodies to bind a specific antigen or agent and mark the antigen/agent for eventual destruction by complement in the absence or presence of phagocytic cells. These assays play a very important role in predicting antibiotic efficacy.

An assay in accordance with this description is the opsonophagocytic assay. Opsonophagocytosis is, briefly, a process whereby an invading cell or microbe, e.g., Streptococcus pneumoniae, is bound by circulating antibody and complement (or complement components). The bound antibody activates the complement cascade resulting in the deposition of certain complement components (e.g. C3b) onto the surface of the microbe. Phagocytic cells or alternative effector cells that have Fc receptors or complement receptors can then engulf and kill the opsonized microbe. Opsonophagocytic assays which are designed to mimic this process have traditionally employed peripheral blood leukocytes (PBLs) as effector cells and generally measure activity by a variety of techniques including radioisotopic, flow cytometric, microscopic, and viability assays; see, e.g., Obaro et al., 1996 Immunol. Lett. 49:83-89; Vioarsson et al., 1994 J. Infect. Dis. 170:592-599; Lortan et al., 1993 Clin. Exp. Immunol. 91:54-57; Kaniuk et al., 1992 Scand. J. Immunol. 36 (Supp. 11):96-98; Esposito et al., 1990 APMIS 98:111-121; Sveum et al., 1986 J. Immunol. Methods 90:257-64; Guckian et al., 1980 J. Infect. Dis. 142:175-190; and Winkelstein et al., 1975 Proc. Soc. Exp. Biol. Med. 149:397-401. A standardized form of this assay was demonstrated wherein culturable phagocytes (differentiated HL-60 cells) were used to measure complement-dependent opsonophagocytic activity in sera from individuals vaccinated with various pneumococcal vaccines; Romero-Steiner et al., 1997 Clin. Diagn. Lab. Immunol. 4(4):415-422. The use of culturable cells (like those of Romero-Steiner) was disclosed to eliminate the need for human donors and decrease the interassay variability that occurs with random PBL donors. Id.

There are alternative means for screening antibacterial agents for effectiveness against the growth and/or viability of a particular microorganism. The serum bactericidal assay (SBA) is one other example of a functionality-based assay capable of evaluating antibodies produced in response to an administered vaccine or bacterial antigen of interest. The purpose of the SBA assay is basically to evaluate whether anticapsular antibodies produced to a bacterial antigen, in combination with complement, are sufficient to confer host protection against invasive disease. This assay has proven particularly effective in the evaluation of vaccines directed against Neisseria meningitidis infection, wherein circulating antibody and complement have been shown to confer host protection to meringococcal disease as early as 1918; Kolmer et al., 1918 J. Immunol. 3:157-175. SBA titer is currently used as a standard by which to determine the effectiveness of proposed vaccine candidates in phase I and II field trials. SBA titer also serves to indicate seroconversion after immunization with currently licensed polysaccharide vaccines; see, e.g., Anderson et al., 1994 Infect. Immunol. 62:3391-3395; Milagres et al., 1994 Infect. Immunol. 62:4419-4424; Zangwill et al., 1994 J. Infect. Dis. 169:847-852. Accordingly, the assay is highly valued in the vaccine and antibiotic area. A standardized SBA assay has been developed at the Center for Disease Control; see Maslanka et al. 1997 Clin. Diagn. Lab. Immunol. 4:156.

Microbial assays such as the bacterial assays mentioned above serve a fundamental purpose in the research evaluation of antimicrobial agents and form a major part of any antimicrobial clinical program. For this reason, it is of great importance to continually design more efficient and optimized means by which to run these and other similar assays. In pursuit of this goal, Applicants have identified the following area in these various types of assays as ripe for improvement.

As one of skill in the art will appreciate, the above assays and microbial assays in general are traditionally run on multi-well plates and generally involve the transfer, and spreading, of an aliquot from each serum dilution of a treated sample to a forum for growth (typically, an agar petri plate). The bacteria or tested microorganism(s) are allowed to colonize on the plate and the appropriate analyses are subsequently carried out so that a determination can be made as to the effectiveness of the purported antimicrobial agent on the growth and/or viability of the microbe. One noted improvement in the art was from spreading aliquots of bacteria, for example, to a more refined “spotting” of bacterial samples (e.g., 5 microliter samples) onto small sections of an agar plate. This allowed for the accommodation of up to 48 samples on a single agar plate.

A common element in these assays, however, remains a limiting factor. Colonies are either counted manually with the aid of a microscope or counted via automated means per agar plate; processes, no doubt, burdened with limited throughput, very labor-intensive, timely, and fraught with opportunities for error. When it is considered that a clinical trial testing a multivalent vaccine may require performing several thousand functional antibody assays, this is a very significant limitation.

A more efficient method for performing the required assays and documenting the results would clearly be advantageous.

### SUMMARY OF THE INVENTION

The instant invention relates to a novel method, for use within serological or screening assays, wherein microbes are grown as colonies on filter membranes in multi-well plates, according to the following process. A sample containing a given microbe (bacteria, for instance) in a liquid (or other transferable) medium, is added to the wells of a multi-well filter plate, for example a Millipore™ Multiscreen™ 96 well filter plate. Excess medium is then removed by a process of vacuum filtration, centrifugation or other suitable means and a nutrient source (in the form of a growth medium) is provided (e.g., THYE broth). Importantly, residual growth medium trapped in or under the filter membrane enables the growth of microbes in discrete colonies on the surface of the filter. Growth of the microbes in this manner allows for the colonies to be stained, imaged, and counted automatically using such automated systems as, e.g., computer and video-based imaging systems. The assay can, thus, be exploited in evaluating the effectiveness of various antimicrobial agents on the growth and/or viability of various microorganisms in a timely and efficient manner.

### BRIEF DESCRIPTION OF THE DRAWINGS

FIG. 1 illustrates opsonophagocytic assay analyses employing the methods described herein wherein the subject bacteria was Streptococcus pneumoniae serotype 14.

FIG. 2 illustrates colony growth obtained upon employing opsonophagocytic assay analyses according to the methods described herein wherein the subject bacteria was Streptococcus pneumoniae serotype 14.

FIG. 3 illustrates colony counts obtained upon employing opsonophagocytic assay analyses according to the methods described herein wherein the subject bacteria was Streptococcus pneumoniae serotype 14.

FIG. 4 illustrates serum bactericidal assay analyses employing the methods described herein wherein the subject bacteria was Neisseria meningitidis serotype C.

FIG. 5 illustrates colony growth obtained upon serum bactericidal assay analyses according to the methods described herein wherein the subject bacteria was Neisseria meningitidis serotype C.

FIG. 6 illustrates, in tabular format, results obtained upon serum bactericidal assay analyses according to the methods described herein wherein the subject bacteria was Neisseria meningitidis serotype C.

FIGS. 7A and 7B illustrates the experimental layout for the analytical validation experiments of the opsonophagocytic assay (Example 5) utilizing the methods described herein. Test samples included in the validation consisted of 48 ELISA negative samples, and three pools of pediatric sera ranging from low/negative to high OPK response as assessed in preliminary runs. The serum pools were tested within each run while the negative samples were evenly divided across runs. Each plate also included (1) four “No Serum” control wells containing bacteria, complement, and HL-60 cells but no antisera; (2) two “Medium Only” control wells, (3) a positive control sera (QC-1) tested at three dilutions, and (4) two specificity controls (QC-1 serum tested with 23F PS and QC-1 serum tested with C-PS).

FIG. 8 illustrates the experimental layout for the specificity studies of the analytical validation experiments. Specificity was assessed by determining the ability of polysaccharides of a known serotype (6B, 9V, 14, 18C, 19F, 23F, and C-Ps at 1 μg/ml) to inhibit killing by positive control antisera (Pool 019 and QC-1 tested at the 1:64 dilution).

FIG. 9 illustrates the results obtained upon utilizing the methods disclosed herein in a serum bactericidal assay to screen hybridoma culture supernatants for the selection of functional monoclonal antibodies that kill Neisseria meningitidis serotype B.

### DETAILED DESCRIPTION OF THE INVENTION

The instant invention relates to a novel method wherein microbes such as bacteria are grown on filter membranes in multi-well plates, enabling the ready analysis of colonies grown thereon. Through the use of this process, the colonies can be readily fixed (i.e., killed), stained, imaged, and counted automatically using such automated systems as, e.g., computer and video-based imaging systems. As such, the instant process avoids the manual counting of colonies grown on agar plates, timely and labor-intensive analyses, and the opportunity for error presented thereby. Also, since microbes in the colonies are killed (fixed) in the process of staining, the method has safety advantages when working with pathogenic microorganisms.

Intrinsic to the instant discovery is the fact that Applicants had realized that residual growth medium trapped in or under a filter membrane is sufficient to enable microbes on the surface of the filter to grow as discrete colonies. The growth obtained on the filter plates is, further, consistent with that seen on agar for purposes of screening downstream antimicrobial activity and, importantly, does not detract from assay precision.

This was not appreciated prior to the instant invention. Filter membranes, to Applicants' knowledge, have been employed in the capture and isolation of microbes, and generally as a stage for various screening and serological assays, such as those carried out in multi-well plates. Upon completion of the reaction(s), however, the bacteria or tested microbe were transferred to a growth medium (e.g., an agar petri plate), and manually counted in order to derive any experimental conclusions. Although devices exist for counting colonies on large (e.g., 100 mm diameter) petri dishes, these devices are not generally amenable to rapid and automated counting of colonies grown in a multi-well (e.g., 48 or 96 well) format. Efficient imaging and counting systems for assays run in multi-well plate format (e.g., the C.T.L. imaging system for ELISPOT assay) have relatively recently become available. These imaging technologies, however, are not capable of enumerating microbial colonies grown on agar due to the irregularities of the agar surface and the difficulties in uniformly plating multiple samples of microbes (e.g., bacteria) on a single plate.

The instant invention bypasses the art in that it provides a means of employing the filter membrane as a forum for microbial growth in a format amenable to automated analyses and avoids, in part, the agar petri plate which does not currently lend itself to efficient automated analyses in a multi-well format. Automated counting of samples in multi-well format has proved more consistent than the manual method, and the assay has proven acceptably rugged to changes in cell passage, operator, plate and counting method; Example 5. There is, further, no evidence that the counting method within the plates affected titer. Notably, individual results tended to be more variable on the agar plate than on the well plate.

The method is, therefore, particularly useful for various serological or screening assays in which determining the effect (microbicidal or microbistatic) of a biological or chemical agent on the number of a particular microorganism of interest microbes is desired. In certain applications, the reaction between the test agent and the microbes can take place in a separate reaction container. The process then generally involves (1) transferring a sample comprising the microbe (e.g., bacteria) in a liquid (or other transferable) medium to the wells of a multi-well filter plate (e.g. a Millipore™ 96 well filter plate or an equivalent thereof (an equivalent thereof being defined as a plate in a multi-well format comprising a filter compatible for use therein)); (2) removing excess media (media other than that captured within and/or under the filter membrane) by a process of vacuum filtration, centrifugation or other means suitable for removing liquid (or other) medium from multi-well plates; and (3) allowing sufficient time for the microbes (e.g., bacteria) to grow into discrete colonies for subsequent analyses. In alternative applications, the assay could feasibly be carried out entirely within and on the microbial filter plate.

Filter plates of use in the instant invention are those suited for use in a multi-well format. The term “filter plates” employed throughout the instant application is to be interpreted as including both specifically crafted “filter plates” in multi-well format as well as simply 96 well plates comprising filters. Particularly preferred are Millipore™ 96 well plates, e.g., the 0.45 μm (pore size) Durapore PVDF filters. Most preferred embodiments of the instant invention employ the Millipore™ MultiScreen™ 96 well plates. It is to be noted that the instant invention is not limited to wells contained within a 96 well format. Any multi-well format suited for ready analysis via automated means is definitely encompassed hereby. Such capabilities are enabled by Applicants' finding that microbes can be manipulated to grow on filter membranes in multi-well format in distinct colonies to an extent comparable for vaccine evaluation purposes to microbes grown on agar.

Filter plates possessing the following characteristics (alone or in combination) have been decidedly preferred for use in the disclosed methods: low levels of protein binding, compatibility with bacterial growth, sterility, and hydrophilicity. Plates possessing all four of these characteristics are most preferred for use within the instant invention. Particularly preferred are Millipore™ HV plates. Opaque plates are further preferred for use within the instant invention as they retard light refraction when undergoing automated analyses. Specifically preferred embodiments of the instant invention employ Millipore™ MultiScreen™ HV 0.45 μm Opaque Sterile Filtration Plates.

Filter plates provide a forum for growth within the well. Bacteria remain on the surface of the filter plates as they are larger than the pore size of the filter membrane. Excess media retained within the multi-well plates is then removed by either vacuum filtration, centrifugation or other means found suitable for removing liquid or alternative transferable medium from multi-well plates. The medium provided to the microbial sample following transfer to the multi-well plate can be any nutrient medium (growth medium) provided to the bacteria or tested microbe(s) following transfer to the filter plates.

The filters, despite the removal process, will retain some medium within or under the filter membrane. Applicants have discovered that this residual medium is surprisingly sufficient to provide adequate nutrients to enable and support the growth of microbes into discrete colonies on the surface of the membrane. Further and importantly, this growth is sufficient for purposes of evaluating the effectiveness of antimicrobial (and, preferably, antibacterial) vaccine candidates. A comparison of the results obtained upon employing bacteria grown on an agar medium with the results obtained upon the use of bacteria grown on filter membranes in the wells of a multi-well plate showed no demonstrable impact of the particular growth forum. Quite to the contrary, analysis in 96 well format proved more consistent, the assay proved acceptably rugged to changes in cell passage, operator, plate and counting method, and there was no evidence that the counting method within the plates affected bacterial titer; Example 5.

Removal of the medium is noted to be important to the disclosed methods. Applicants have found that medium beyond residual medium (that trapped within or under the filter) is undesirable. Insufficient removal results in growth of the microbe(s) as a homogeneous suspension, rather than as discrete colonies required for accurate enumeration and analysis. More, it is preferred that the medium is removed in such a manner as to increase the contact of microbes present in the sample with the filter membrane (e.g., as in vacuum filtration and centrifugation, where the liquid is funneled downwardly through the membrane).

As indicated above, following removal of medium from the wells, the microbe(s) are incubated for an amount of time sufficient to permit growth into discrete colonies for subsequent analyses; preferably, 14-18 hours, but generally dependent on the particular microorganism. Preferably, the plates comprising the microbes are kept hydrated. This can be accomplished through a number of means such as incubation in a humidified environment such as a water-jacketed humidified incubator or by covering the filter plate in, for instance, a Ziploc bag. Any suitable alternative serving to accomplish this same function is also encompassed hereby.

An interesting feature of the instant method is that the colony size is limited by the available nutrients trapped within or under the filter membrane. Thus, when there are few colonies, the individual colonies are relatively large, whereas when there are many colonies, the individual colonies are smaller. The benefit of this particular finding is that, in certain applications, up to approximately 300 colonies per well can be enumerated in an individual well, rendering the dynamic range of the method wider than that for the previously employed agar-based methods in which the maximum number of colonies discernable after “spotting” of 5 microliter samples is approximately 70.

After some time during which the microbe(s) are allowed to grow into discrete colonies on the filter plate, the colonies are fixed and stained; a preferred stain of which is Coomassie blue, but which can be any agent capable of providing a means for specifically highlighting an object for detection by the visualization, imaging and/or enumeration system employed. Staining in general terms enables ready visualization of the colonies by the system employed. The colonies are then analyzed. This is accomplished with any device suited for analysis of 96 well plates, or whichever multi-well format is utilized. Preferred for use in the instant invention is any computer-assisted video imaging and analysis system. In a particularly preferred embodiment, the computer-assisted imaging and analysis system is the ImmunoSpot™ Analyzer offered by C.T.L. (Cleveland, Ohio), or a similar imaging system offered by, for instance, Resolution Technology, Inc. (Columbus, Ohio).

Applicants believe the methods disclosed herein are widely applicable to various microorganisms, e.g., bacteria, viruses, algae, fungi (e.g., Candida albicans and Aspergillus) and protozoa. Samples can be obtained from any of a number of sources. For purposes of exemplification, the bacteria can be isolates of Streptococcus pneumoniae, Neisseria meninigitidis, E. coli, Staphylococcus aureus, Bacillus anthracis, or any gram-positive or gram-negative bacteria.

The bacteria or other microbe(s) are generally contained in a liquid (or other transferable) medium. A medium containing a nutrient source (a growth or nutrient medium) must be provided. Broth as a nutrient medium is particularly preferred. Particular embodiments of the instant invention employ Todd-Hewitt (“TH”) yeast extract (“THYE”) broth. In preferred embodiments, said broth is employed for the growth of Streptococci. Alternative embodiments employ tryptic soy broth (“TSB”). In specific embodiments, said TSB broth is utilized for the growth of Neisseria and E. coli. Alternative broth media suitable for the growth of the microorganism of interest can be used. In the examples provided, 100 μl per well was added to the Millipore™ 96 well HV plate.

Using the disclosed methods, one of skill in the art can evaluate the efficacy of any particular antimicrobial agent. The antimicrobial agent of interest which can be any compound (e.g., antibiotic or microbistatic agent) or biological (e.g., antiserum) having an impact on the growth and/or viability of the microbe(s) of interest is placed in contact with the microbial sample and left for a period of time. Transfer of the microbial sample to the multi-well plate can take place before or following contact with the antimicrobial agent. In the situation wherein the microbial sample is brought into contact with the antimicrobial agent prior to transfer, this contact takes place in a different medium, for instance another well or an agar plate. In specific embodiments, the microbial samples to be analyzed are transferred from mediums wherein various functional assays, such as opsonophagocytic and serum bactericidal assays were run. In one specific embodiment, prior to transfer to the filter plate, the bacteria are “preopsonized” (i.e., placed in contact with antibody (see, e.g., Example 2D)); and placed in contact with complement (or the active components thereof) and differentiated HL-60 cells (or any cells capable of clearing the marked bacteria, e.g., polymorphonuclear leukocytes). Antibody which can be utilized in these experiments can be derived from an individual or animal which was administered the microbe or an effective antigen of same. Antibodies specific to the bacteria or microbe of interest contained within the sample can also be obtained conmmercially or via generation outside of the human (e.g., in rabbit). In another embodiment of the instant invention, prior to transfer to the filter plate, the bacteria are preopsonized and then placed in contact with complement (or active components thereof); (see, e.g., Example 4D).

Antimicrobial agents which can be evaluated via the utilization of the methods disclosed herein can be selected from any compound (e.g., antibiotic or microbistatic agent) or biological (e.g., antiserum) that impacts the growth and/or viability of the specific microbe of interest. Hybridoma culture supernatant can be screened for the production of monoclonal antibodies effective against a microbe of interest.

The following non-limiting Examples are presented to better illustrate very specific embodiments of the present invention. As one of ordinary skill in the art will appreciate, there are numerous ways in which one can carry out the various methods disclosed herein and accomplish the same or similar results.

### EXAMPLES

The required precautions and procedures outlined for compliance with OSHA's Blood Borne Pathogen Standard should be followed when handling human blood products.

### Example 1

### Materials and Equipment for the Opsonophagocytic Assay

A. Materials

1. 96-Well Cell Cluster Plates (U-bottom) (Costar, cat no. 3799)
2. Reagent Reservoirs, (Fisher Scientific, cat no. 13-681 -101)
3. Sterile (0-200 μl) pipette tips
4. Tips for Matrix Pipette (Matrix Technologies, 0-850 μl, cat no. 8052)
5. Serology disposable pipettes, sterile (2, 5, 10, 25, 50 mL volume)
6. Hemocytometer or equivalent (e.g. Kova Glasstic Slide (Fisher Scientific, cat no. 22-270141))
7. Centrifuge tubes: 15 ml and 50 ml conical polystyrene centrifuge tubes
8. Millipore™ MultiScreen™ filtration plate, HV, Opaque sterile, cat no. MHVBS4510
9. Whatman filter paper No. 5, circles, 150 mm diameter, cat no. 1005 150
10. CD-Recordable Disks
11. Ziploc Bags for HV plate overnight incubation
12. HV plate storage, vacuum-sealable bags
   B. Equipment
1. CO₂ Incubator set at 37±2° C., 5±1% CO₂
2. Centrifuge for cell washing (Jouan CR422)
3. Matrix Pipette (Matrix Technologies, 15-850 μl, cat no. 2014)
4. Single channel pipettes: 1-10 μl, 20-100 or 200 μl, and 100-1000 μl
5. Multi channel pipettes (5-50 μl, 50-200 μl)
6. Inverted microscope with 1× and 10× objectives
7. Compound microscope
8. Horizontal rotator shaker (up to 220 rpm range) Thermolyne RotoMix type 50800
9. Colony counting system: ImmunoSpot™ counter (Cellular Technology Limited (C.T.L.), Cleveland, Ohio)
   C. Reagents
1. Gelatin (2% solution) (Sigma, cat no. G1393)
2. Hank's Buffered Saline Solution (HBSS) (Gibco-BRL Life Technologies, cat no. 14025-092)
3. Hank's Buffered Saline Solution (HBSS) without Ca2+, Mg2+ (Gibco-BRL Life Technologies, cat no. 14175-095)
4. Todd-Hewitt Broth (Becton Dickinson, cat no. 8117360, Fisher Scientific, cat no. B11736)
5. Yeast Extract powder (#28926), Difco
6. 3-4 week old rabbit complement, sterile (Pel. Freez, code no. 31061)
7. HL-60 cells differentiated as described in Romero-Steiner et al., 1997 Clin. Diagn. Lab. Immunol. 4(4):415-422
8. 0.4% trypan blue solution in PBS, pH 7.2
9. S. pneumoniae bacteria (frozen aliquots of various serotypes: 1, 4, 6B, 9V, 14, 18C, 19F, and 23F)
10. S. pneumoniae capsular polysaccharides (various serotypes: 4, 6B, 9V, 14, 18C, 19F, and 23F)
11. 0.01% Coomassie blue prepared in 45% methanol, 45% water and 10% acetic acid
12. Coomassie blue diluent: 45% methanol, 45% water and 10% acetic acid
13. Sterile water (RCM-66)
   D. Media
1. Todd-Hewitt Broth with 0.5% yeast extract, 50 ml aliquot, autoclaved
2. Gelatin (0.1%) in HBSS with Ca⁺⁺, Mg⁺⁺ (“Opsono buffer”)

### Example 2

### Procedure for Opsonophagocytic Assay

Plate Layout for OPK Assay (Serotype 6B)

| Sample 1 | | Sample 2 | | Sample 3 | | Sample 4 | | Sample 5 | | Controls | |
|---|---|---|---|---|---|---|---|---|---|---|---|
| 1 | 2 | 1 | 2 | 1 | 2 | 1 | 2 | 1 | 2 | 1 | 2 |

| | | | | | |
|---|---|---|---|---|---|
| 1:8 | 1:8 | 1:8 | 1:8 | 1:8 | Cells + C′ |
| 1:16 | 1:16 | 1:16 | 1:16 | 1:16 | (no serum) |
| 1:32 | 1:32 | 1:32 | 1:32 | 1:32 | QC serum (2X titer) |
| 1:64 | 1:64 | 1:64 | 1:64 | 1:64 | QC serum (1X titer) |
| 1:128 | 1:128 | 1:128 | 1:128 | 1:128 | QC serum (0.5X titer) |
| 1:256 | 1:256 | 1:256 | 1:256 | 1:256 | QC sera + 6B Ps* |
| 1:512 | 1:512 | 1:512 | 1:512 | 1:512 | QC sera + C-Ps* |
| 1:1024 | 1:1024 | 1:1024 | 1:1024 | 1:1024 | Medium alone |

A. Test Sample Dilutions

Note: Serum samples are generally tested without pre-dilution. A minimum of 40 μl is recommended needed to test a serum sample (in duplicate) for opsonic activity against a single serotype of pneumococci. Per specific experiment needs, sera may be heat-inactivated (56±2° C., 30±5 minutes) prior to testing, and repeat cycles of freezing and thawing should be avoided. Samples are run in duplicate, so A1 and A2 are the same serum, A3 and A4 from a second serum sample, and so on until wells All through A12 are reached. These last two columns are used for the complement (“C”′) controls and for Quality Control (“QC”) tests of assay validity (see plate template above).
1. A sterile reagent reservoir was filled with Opsono buffer and a multi-channel pipette was used to add 10 μl of Opsono buffer to all wells in columns 1-10 except Row A.
2. 20 μl of undiluted serum was aliquotted into the appropriate wells in Row A according to the plate set-up template.
3. Two-fold serial dilutions of the samples were made using a multichannel pipetter adjusted to 10 μl by first transferring 10 μl from wells A1 to A10 in the first row to the corresponding wells in Row B; avoiding air bubbles.
4. The contents of the wells were carefully mixed by pipetting up and down ˜5 times.
5. 10 μl from the second row (Row B) was transferred to the third row (Row C) and step 4 was repeated. This process was continued on through Row H. The excess 10 μl from Row H was discarded into the waste.
6. All wells now contained 10 μl. Note: Once the remainder of the reagents are added, Row A will become a 1:8 dilution. Row B=1:16, row C=1:32, row D=1:64, row E=1:128,row F=1:256,row G=1:512 and row H=1:1024. Columns 11-12 will contain pre-diluted QC test sera and controls; see below.
7. 10 μL of opsono buffer was added to all wells in columns 1-10 to bring the total volume to 20 μL per well. Note: Buffer was not added to the wells in column 11 and 12 at that time.
   B. Plate Quality Control

Each plate had its own controls for assay validity. These controls were set up in columns 11 and 12:
1. Complement control wells were (A11, A12, B11, B12): 20 μl of Opsono buffer was added to make the volume equal to the volume of the test sample wells.
2. Positive QC sera controls: QC sera were prepared by pooling serum from adult humans who had previously been vaccinated with one or more doses of pneumococcal vaccine. The serum pools were pre-tested in the OPK assay to determine the endpoint titer against each serotype. The QC serum was included in each assay plate at three dilutions expected to bracket the pre-determined titer for a given serotype. QC serum in appropriate wells was diluted to approximate dilutions of 2× (C11, C12), 1×(D11, D12), and 0.5×(E11, E12), the predetermined endpoint titer for that lot of QC serum. 10 μL of Opsono buffer was added to these wells, for a final volume of 20 μL.
3. Specificity Controls:
   Serotype specific control (F11, F12): These wells contained 10 μl of known titer sera which approximately represents the 2× concentration of OPK titer and 10 μg of serotype-specific polysaccharide at 8 μg/ml.
   Serotype non-specific control (G11, G12): These wells contained 10 μl of known titer sera which give the 2× concentration of OPK titer and 10 μl of non-specific bacteria cell wall polysaccharide (C-Ps) at 8 μg/ml.
4. Medium alone control (H11, H12): 30 μl of Opsono buffer was added to these wells (H11 and H12).
5. For an assay plate(s) to be valid, it was noted that (1) the positive control QC serum must be positive (i.e., ≧50% inhibition) at least at one of three dilutions tested (e.g. 1:320, 1:640, or 1:1260; C11, C12; D11, D12; AND E11, E12, respectively); (2) The specificity control wells (quality control (“QC”) serum (F11, F12)+homologous polysaccharide (“PS”); G11, G12) must be negative (i.e., <50% inhibition); and (3) the average number of colonies across the four “No Serum” (complement control) wells (A11, A12, B11, B12) must be ≧25.
   C. Harvesting Differentiated HL-60 Cells
1. Flasks were gently swirled to distribute cells and the contents of each flask were then poured into 50 mL centrifuge tubes.
2. The cells were centrifuged at 160×g for 10 minutes at room temperature. (Note: this step was started before adding bacteria to the plate for preopsonization). Preopsonization Steps 1-4 (Example 2D) were performed before proceeding to the next step.
3. Supernatant was poured into sterile bottles for disposal and the remaining medium was discarded by inverting tube with a single motion and draining excess medium by keeping the tube inverted until the supernatant was removed. Note: Differentiated HL-60 cell medium contains DMF and should be disposed as chemical waste.
4. The HL60 cell pellet was resuspended from every 100 mL of original culture that was used in 40-50 ml of Hanks buffer (room temperature) without Ca⁺⁺, Mg⁺⁺ and phenol red. Note: If 200 mL of original cell culture are used, use 80-100 mL of the Hank's Buffer without Ca⁺⁺, Mg⁺⁺ and phenol red.
5. An aliquot of cells was removed and viable cell count was determined by staining cells with Trypan blue dye and counting cells using a hemocytometer or equivalent tool.
6. Meanwhile, cells were centrifuged at 160×g for 10 minutes at room temperature and supernatant was removed as in step 3 of Example 2C. Note: Step 1 of Example 2E was performed at this point to allow the baby rabbit serum complement to thaw.
7. The H60 cell pellet was resuspended in opsono buffer at 1×10⁷ cells per ml. These cells were then ready to use in the assay, using 4 ml per plate. They were kept at room temperature until their use. Note: A slight (e.g. at least 1 mL) excess of cells was purposely present for the assay. Note: only plastic pipettes were used. Cells attach to glass. Hanks was gently pipetted up and down 3 to 4 times only, and the pellet was dispensed slowly.
   D. Preopsonization
1. A bacteria frozen stock of the desired strain from the −70° C. freezer was allowed to thaw at room temperature, and used immediately; otherwise, the thawed bacteria need be kept on ice until ready to make dilution but no longer than 1 hour.
2. The contents of the vial were mixed by slowly pipetting about one-half the volume of medium in the vial up and down 3 to 5 times; avoiding the introduction of air into the medium. Note: Do not vortex. An initial dilution was made of 1:10 as well as subsequent dilutions as needed to reach the desired final concentration. Dilutions were made with Opsono buffer (usually in 2-3 steps, e.g., 1:10, then, 1:100 and, finally, 1:40 to make a 1:4000 dilution) in order to adjust the number of bacteria to ˜1,000 cfu/10 μl. Note: (Frozen aliquots of S. Pneumoniae were pre-tested to determine the appropriate dilution.)
   Note: Each plate requires ˜1 ml of diluted bacteria. Dilution varies according to each frozen strain and lot number. Make sure you have a slight (e.g. at least 1 mL) excess of cells for the assay.
3. A multichannel pipette set to 10 μl was used to add the diluted bacterial suspension to each of the 96 wells in a microtiter plate, except for the “medium alone” wells, H11 and H12. A sterile reagent reservoir was used to hold the diluted bacterial suspension. Where multiple plates were being run, the bacteria dilution was mixed by repeated aspiration and expulsion with a pipette, between additions to successive plates.

Volume in Each Well at this Point:

| | Titer test | C′ control | Specificity | Med. alone |
|---|---|---|---|---|
| Undiluted or | 10 μl | — | 10 μl | — |
| diluted serum: | | | | |
| Opsono buffer: | 10 μl | 20 μl | — | 30 μl |
| Polysaccharide | — | — | 10 μl | — |
| (specif.'y. control): | | | | |
| Bacterial | 10 μl | 10 μl | 10 μl | — |
| suspension: | | | | |
| Total: | 30 μl | 30 μl | 30 μl | 30 μl |

4. The plates were covered with their lids and the microtiter plates were incubated on a horizontal rotator shaker at 200 rpm at 37±2° C., 5±1% CO₂ for 15±2 min. This step allowed the pre-opsonization of bacteria with diluted sera.
   E. Complement Addition
1. Frozen stocks of complement were stored at −70° C. The vials of baby rabbit serum were thawed immediately before use. Note: The complement should be at ˜4° C. before addition to plates. One vial of baby rabbit serum that contains at least 1 mL volume, per assay plate was thawed; and one additional vial was thawed to have a slight excess, to cover multiple plate usage.
2. Following the preopsonization, 10 μl of complement source (Baby Rabbit Serum) was added to all 96 wells using a multi-channel pipetter.
3. When adding the complement, a 1 ml vial was enough for one microtiter plate. All the vials necessary for the number of plates being run were thawed and combined in a sterile reagent reservoir.
4. The vials were pooled in a sterile reagent reservoir and the complement was quickly added to all wells.
   F. Phagocytosis with HL-60 Cells
1. 40 μl of the washed resuspended cells (at 1×10⁷ ml) was added into each well of a microtiter plate for the opsonophagocytic assay.
   Note: The total number of cells added to each well were and should be 4×10⁵ in 40 μl. Each 96 well plate requires 4 ml. A sterile reagent reservoir was used to hold the cell suspension.

Volume in Each Well at this Point:

| | Titer test | C′ control | Specificity | Medium alone |
|---|---|---|---|---|
| Preopsonization | 30 μl | 30 μl | 30 μl | 30 μl |
| Baby rabbit serum | 10 μl | 10 μl | 10 μl | 10 μl |
| Differentiated | 40 μl | 40 μl | 40 μl | 40 μl |
| HL-60 cells | | | | |
| Total volume | 80 μl | 80 μl | 80 μl | 80 μl |

2. The lids were replaced and the microtiter plates were incubated on a horizontal rotator shaker at 220±20 rpm at 37±2° C., 5±1% CO₂, for a period of 45±5 minutes.
   G. Viable Bacteria Enumeration
1. Sterile water (RCM-66) was added to Millipore™ 96 well HV plate, 200 μl per well.
2. The contents in the wells from the opsono reaction were carefully mixed (4-6 times) and the desired volume of sample (e.g. 5 or 10 μl) from each well was transferred into the corresponding wells of the HV plates using a multichannel pipette; this was done column by column.
3. Filtration: The HV plate was placed on the Millipore™ MultiScreen™ filtration system and the pump was turned on. Note: Make sure meter is showing 10±2 in. Hg-vac. The plate contents were filtered through the membrane by pushing down the plate with the lid to the base of the unit until the plate was fully emptied and no liquid remained visible.
4. THYE broth (room temperature) was added to the Millipore™ 96 well HV plate; 100 μl per well.
5. Filtration: Step 3 was repeated.
   Note: Filtration can happen very fast (within 5-10 seconds). Therefore, it is recommended that one monitor the filtration carefully to prevent over-drying of the plate. Note that too much broth remaining in the wells will give high background and poor quality staining. Remove plate from the manifold immediately after most of the broth is drawn through the wells, but avoiding over-drying and over-filtration. Carefully monitor wells during filtration to observe when well contents have been aspirated, as discernable by the disappearance of liquid from the outside edges of the wells. Remove plate immediately after this occurs.
6. After filtration, the base of the HV plate unit was pat dry on a paper towel.
7. The plate was wrapped with plastic film to seal all moisture inside of the plate.
8. The wrapped plate was inserted in a sealable, plastic Ziploc bag and incubated overnight at 37±2° C., 5±1% CO₂, for a period of 20-24 hours. The plates were placed in an upright position in the incubator.
   H. Colony Staining and Counting
1. 0.01% Coomassie blue was filtered with Whatman No. 5 filter paper to remove undissolved Coomassie blue precipitates. Approximately 5 ml of stain was required for each HV plate. Only fresh-filtered stain was used for HV plates.
2. Colony staining: The HV plate was placed on the Millipore™ filtration system without pushing down (Note the plate was off the vacuum). The pump was turned on. Note: Make sure meter is showing 10±2 in. Hg-vac. 50 μl per well of filtered Coomassie blue was added to the plate using Matrix Multichannel pipette. The plate was only stained for 15 to 30 seconds; then, the plate was pushed down onto the filtration manifold to empty the contents by vacuum filtration.
3. Destaining: Once the plate was fully emptied, 100 μl per well Coomassie blue diluent was immediately added to each well and the plate was kept under the vacuum until no liquid was visible. This step removed most non-specific staining.
4. The base of the plate was pat dry on a paper towel or equivalent and the base was separated from the 96 well multiscreen plate.
5. The plate was dried upside down in a Biosafety hood with circulating air, until wells were completely dried; for approximately 10 minutes.

6. ImmunoSpot™ counter setting for colony counting:

| | Standard setting |
|---|---|
| Sensitivity: | 180 |
| Spot size: | 0.03 |
| Separation tolerance: | 8.0 |
| Diffuse counting is: | on |
| Diffuseness: | 20 |
| Overdeveloped area handling is: | off |
| Color system: | Blue |
| Detailed counting is: | off |
| Background balance is: | on |
| Background balance: | 110 |
| Hole filling is: | on |

Note: ImmunoSpot™ settings were calibrated and adjusted at 3 month intervals by comparison with counts obtained by human readers.
7. The plate was scanned in the imaging system, the image was printed and the data was counted. The image data were saved as tiff files on CD-R disk.
   I. OPK Titer Calculations

The phagocytic titer is the reciprocal of the serum dilution with at least 50% killing, when compared to the average growth in the complement control wells. Occasionally, sera with high titers need to be re-tested at higher initial dilutions than 1:8 to determine the phagocytic titer. Serum samples with phagocytic titers <8 are reported as a titer of 4 for purposes of data analysis.
% Killing=No Serum Control Avg CFU—Test Sample Avg CFU/No Serum Control Avg CFU—Medium Only Avg CFU×100%
J. Experimental Results

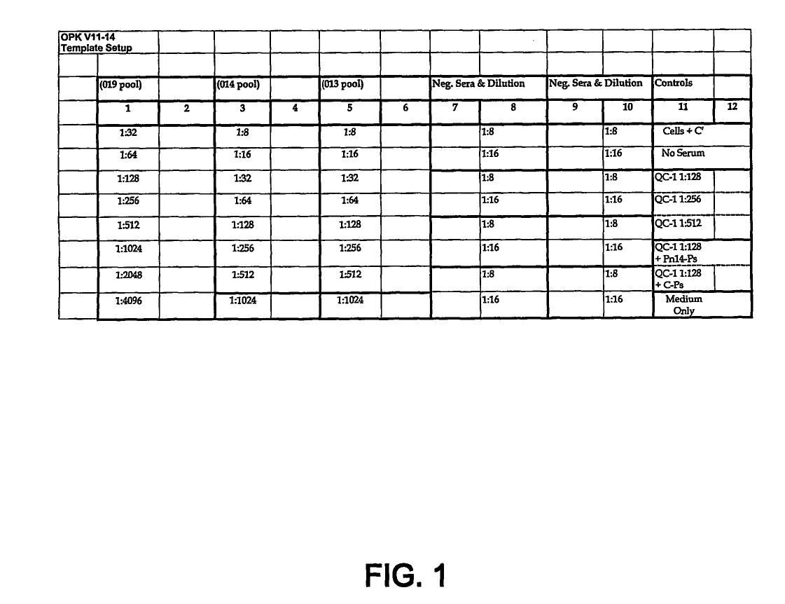

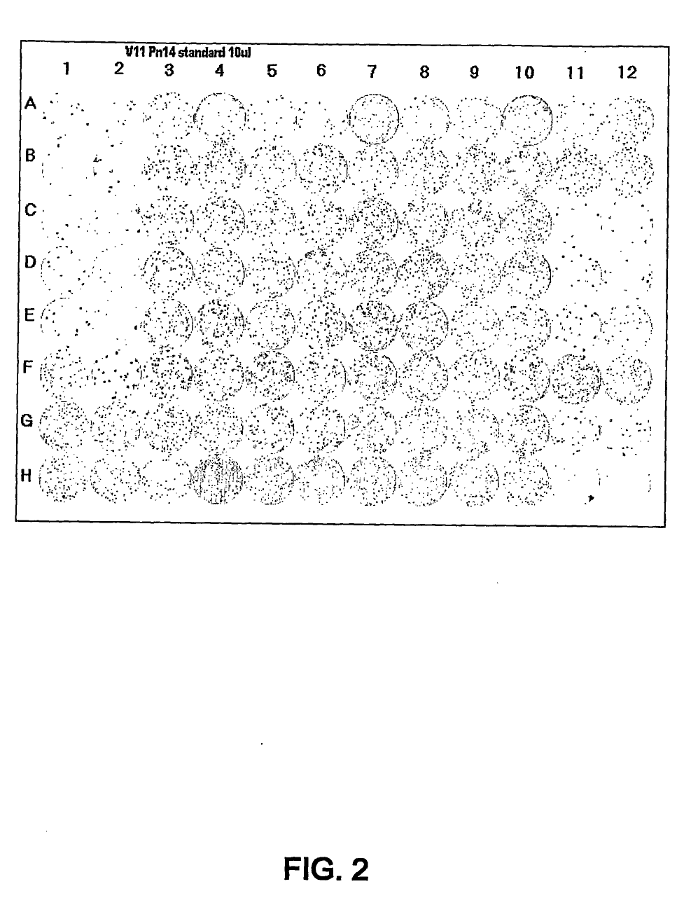

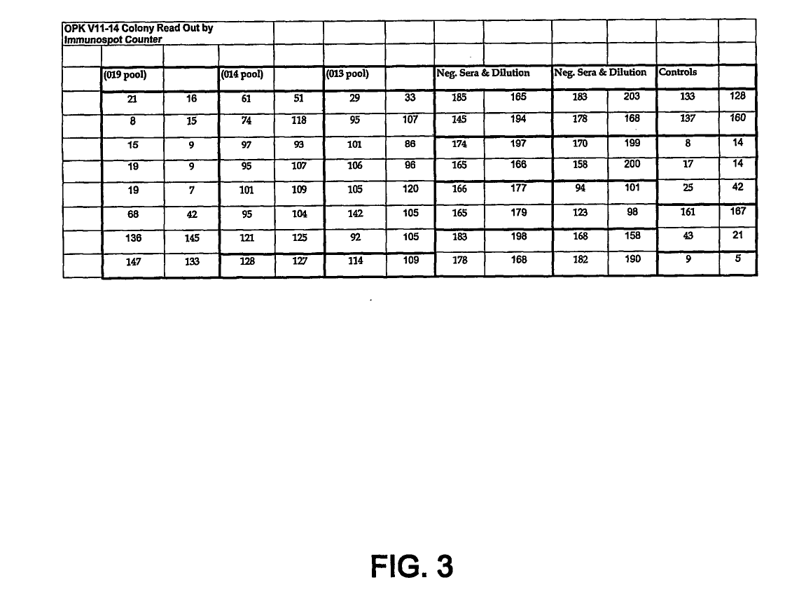

OPK assays were individually run with bacterial samples of S. pneumoniae serotypes 6B, 9V, 14, 18C, 19F and 23F. Bacterial growth or lack thereof (due to the effect of an antibiotic substance) was able to be detected and analyzed within the multi-well plates. FIG. 1 shows the basic assay template set-up utilized to run the experiment with serum samples of S. pneumoniae serotype 14. FIGS. 2 and 3, respectively, show the colony read-out obtained from the wells both pictorially and numerically.

### Example 3

### Materials and Equipment for Serum Bactericidal Assay

A. Materials

1. 96-Well Cell Cluster Plates (Flat-bottom cell culture cluster, polystyrene) (Costar, cat no. 3595)
2. Reagent Reservoirs, (Fisher Scientific, cat no. 13-681-101)
3. Sterile (0-200 μl) pipette tips (Matrix)
4. Tips for Matrix Pipette (Matrix Technologies, 0-850 μl, cat no. 8052)
5. 14 ml polypropylene round-bottom tube, 17×100 mm style (Becton-Dickinson, Falcon cat no. 352059)
6. 5 ml polystyrene round-bottom tube, 12×75 mm style (Becton-Dickinson, Falcon cat no. 352058)
7. Millipore™ MultiScreen™ filtration plate, HV, Opaque sterile 0.45 μm hydrophilic, Durapore membrane, cat no. MHVBS4510
8. Whatman filter paper No. 5, circles, 150 mm diameter, cat no. 1005 150
9. CD-Recordable Disks, Imation (ISG cat no. IMN-41181)
10. Fisherbrand Autoclave Bags for HV plate overnight incubation (8.5″×11″, Fisher Scientific #01-8151)
   B. Equipment
1. CO₂ Incubator set at 37±2° C., 5±1% CO₂
2. Matrix Pipette (Matrix Technologies, 15-850 μl, cat no. 2014)
3. Single channel pipettes: 1-10 μl, 20-100 or 200 μl, and 100-1000 μl (Lab Systems)
4. Multi channel pipettes (5-50 μl, 50-200 μl) (Lab Systems)
5. Horizontal rotator shaker (up to 220 rpm range) Thermolyne RotoMix type 50800
6. Colony counting system: ImmunoSpot™ counter (C.T.L., Cleveland, Ohio)
7. Millipore MultiScreen Separation System (Millipore, cat no. MAVM 096 0R)
8. Vacuum pump unit: VakuumSystem type ME 2S1 (Vacuubrand GMGH+Co).
   C. Reagents
1. Glucose (Aldrich, cat no. 25307-3)
2. Dulbecco's PBS (D-PBS) (Sigma, cat no. D8537)
3. Tryptic Soy Broth (TSB): (Difco, Becton Dickinson cat no. 290612), sterile, 100 ml/bottle.
4. 3-4 week old rabbit complement, sterile (Pel. Freez, code no. 31061)
5. Neisseria Meningitidis bacteria (frozen aliquots of various serotypes: A, B, C, Y, and W-135): Originally obtained from Dr. Sandra Steiner, Centers for Disease Control and Prevention.
6. Neisseria Meningitidis capsular polysaccharides (various serotypes: C, Y, and W-135) 1 mg/ml in sterile water. Stored at—80 C. Merck Research Labs.
7. 0.01% Coomassie blue prepared in 45% methanol, 45% water and 10% acetic acid. Stored at room temperature for up to 3 months.
8. Sterile water (RCM-66)
   D. Media
1. 10% glucose: prepared in sterile distilled water (RCM-66), 0.45 μm filtered.
2. D-PBS-0.1% glucose (D-PBS-Glc): Dulbecco's PBS, 0.1% glucose, 0.45 μm filtered. Prepared by 1:100 dilution of 10% glucose in D-PBS.

### Example 4

### Procedure for Serum Bactericidal Assay

Plate Layout for SBA Assay (Serotype C)

Serogroup C bacteria

| Sample 1 | | | | Sample 2 | | | | Sample 3 | | | |
|---|---|---|---|---|---|---|---|---|---|---|---|
| Pre | | Post | | Pre | | Post | | Pre | | Post | |
| 1 | 2 | 3 | 4 | 5 | 6 | 7 | 8 | 9 | 10 | 11 | 12 |

| | | | | | | |
|---|---|---|---|---|---|---|
| A | 1:4 | 1:4 | 1:4 | 1:4 | 1:4 | 1:4 |
| B | 1:8 | 1:8 | 1:8 | 1:8 | 1:8 | 1:8 |
| C | 1:16 | 1:16 | 1:16 | 1:16 | 1:16 | 1:16 |
| D | 1:32 | 1:32 | 1:32 | 1:32 | 1:32 | 1:32 |
| E | 1:64 | 1:64 | 1:64 | 1:64 | 1:64 | 1:64 |
| F | 1:128 | 1:128 | 1:128 | 1:128 | 1:128 | 1:128 |
| G | 1:256 | 1:256 | 1:256 | 1:256 | 1:256 | 1:256 |
| H | PCS 1:16 | PCS 1:16 + | C′ control | C′ control | PCS 1:16 + bacteria | Medium |
| | | Mn C- Ps 500 | | | only | control |
| | | ng/ml | | | | |

PCS: positive control serum

A. Plate Quality Control

Each plate should have its own controls for assay validity. These controls were set up in Row H (wells H1 to H12):

Positive serum was diluted in a separate tube to give a final dilution of 8 times the predetermined endpoint titer for positive control serum. When this serum was added to the wells on the plate, the final concentration was 4 times the predetermined endpoint titer. A minimum of 150 μl of diluted positive control was needed for each plate.
1. Positive sera control wells (H1 and H2): These wells contained 25 μl of positive control serum at a final concentration of 4 times the pre-determined SBA titer.
2. Serotype specific control (H3 and H4): These wells contained 25 μl of positive control serum at a final concentration of 4 times the pre-determined SBA titer and 10 μl of serotype-specific polysaccharide at 3 μg/ml (final on plate concentration is 500 ng/ml).
3. Complement control (i.e., no serum) wells (H5, H6, H7, H8): 25 μl of D-PBS-Glc buffer was added.
4. Serum and bacteria only wells (H9 and H10): 12.5 μl of D-PBS-Glc buffer was added.
5. Medium alone control (H11 and H12): 50 μl D-PBS-Glc buffer was added to these wells (H11 and H12).

Note: When all components were added, the volume of each reagent in Row H control wells was as follows: (complement and bacteria were added in later steps which are also listed here for reference)

| | Positive | Specificity | C′ control | Serum bacteria | c Medium |
|---|---|---|---|---|---|
| | serum | control | (bacteria + C′) | (serum control) | control |
| D-PBS-Glc buffer | — | — | 25 μl | 12.5 μl | 50 μl |
| Polysaccharide | — | 10 μl | — | — | — |
| Diluted serum | 25 μl | 25 μl | — | 25 μl | — |
| C′ | 12.5 μl | 12.5 μl | 12.5 μl | — | — |
| Bacteria | 12.5 μl | 12.5 μl | 12.5 μl | 12.5 μl | — |

Reagents were Added in the Following Order:

i) D-PBS-Glc buffer; ii) polysaccharide; and iii) diluted positive control serum. After adding the above reagents to the control wells, work should be carried out on the test wells.
Serial dilutions were made of the test serum.
Then dilutions were made of the bacteria.
Bacteria were diluted to the desired dilution.
After making dilutions, an appropriate volume of bacteria at the final dilution was taken out and mixed with equal volume of complement. For one plate, at least 1.2 ml diluted bacteria and 1.2 ml complement is recommended.
25 μl of bacteria and complement mixture was added to the test and control wells except the “serum and bacteria only” wells and the “medium control” wells.
12.5 μl bacteria at final dilution was added to the “serum and bacteria only” wells.
For one plate, 50 μl diluted bacteria was needed.
   B. Test Sample Dilutions

Note: Serum samples were generally tested without pre-dilution. A minimum of 25 μl was determined to be needed to test a serum sample (in duplicate) for bactericidal activity against a single serotype of meningococci. Per specific experiment needs, sera may be heat-inactivated (56±2° C., 30±5 minutes) prior to testing. Repeat cycles of freezing and thawing should be avoided. Serum samples were tested at a starting dilution of 1:4 (in plate final dilution) and diluted in a two-fold dilution scheme. Samples were run in duplicate, so A1 and A2 are the same serum, A3 and A4 from a second serum sample, and so on until all wells of the entire row are occupied.
1. A sterile reagent reservoir was filled with D-PBS-Glc buffer and a multi-channel pipette was used to add 25 μl of D-PBS-Glc buffer to all wells in columns 1-12 except Row A and Row H. Wells in Row H were used for the C′ control, medium control and other controls. (See plate template).
2. 37.5 μl of D-PBS-Glc buffer was added to all wells in Row A.
3. 12.5 μl of undiluted serum was aliquotted into the appropriate wells in Row A according to the plate set-up template.
4. Two-fold serial dilutions of the samples were made using a multichannel pipetter adjusted to 25 μl by first transferring 25 μl from wells A1 to A12 in the first row to the corresponding wells in Row B; avoiding air bubbles.
5. The contents of the wells were carefully mixed by pipetting up and down ˜5 times.
6. 25 μl was transferred from the second to the third row and step 5 was repeated. This process was continued on through Row G. The excess 25 μl from Row G was discarded into the waste.
7. All wells now contained 25 μl. Note: Row A becomes a 1:4 dilution once the remainder of the reagents are added. Row B=1:8, Row C=1:16, Row D=1:32, Row E=1:64, Row F=1:128, and Row G=1:256. Row H contains controls (See plate template above).
   C. Preparation of Bacteria
1. A bacteria frozen stock of the desired strain from the−70±15° C. freezer was allowed to thaw at room temperature and used immediately; otherwise, the thawed bacteria need be kept on ice until ready to make the dilution, but no longer than 1 hour.
2. The contents of the bacteria vial were mixed by slowly pipetting about one-half the volume of medium in the vial up and down 3 to 5 times; avoiding the introduction of air into the medium. Note: Do not vortex. An initial dilution was made of 1:10 as well as subsequent dilutions needed to reach the desired final concentration. Dilutions were made in D-PBS-Glc buffer (usually in 2-3 steps, e.g., 1:10, then, 1:100 and, finally, 1:30 to make a 1:3000 dilution) in 5 ml round-bottom tubes. The number of bacteria were adjusted to ˜1,000 cfu/12.5 μl, or ˜50-150 cfu/5 μl aliquot from the total volume in the complement control well (usually 10³ to 10⁴ dilution from working stock if the working stock was prepared when bacteria grew to a density of OD600 around 0.4 to 0.5).
   (Frozen aliquots of N. meningitidis were pre-tested to determine the appropriate dilution.) Note: Each plate requires ˜1.2 ml of diluted bacteria. Dilution varies according to each frozen strain and lot number. Make sure you have a slight (e.g. at least 0.3 ml) excess of diluted bacteria for the assay.
   D. Bacteria-Complement Addition
1. Frozen stocks of complement were stored at −70° C. The vials of baby rabbit serum were thawed immediately before use. Note: The complement should be at ˜4° C. before addition to plates. Two vials of baby rabbit serum that contains at least 1.2 mL combined volume, per assay plate were thawed. All the vials of baby rabbit serum were combined in a sterile 14 ml tube. Once the complement was pooled, it was added to the plates within 5 minutes after removing the vials from the ice.
2. The appropriate volume of bacteria at the final dilution was taken out, and the bacteria suspension was mixed with an equal volume of complement in a fresh sterile 14 ml tube. A multichannel pipetter set to 25 μl was used to add the bacteria-complement mix to each of the 96 wells in a microtiter plate except the “serum and bacteria only” control wells and “medium control” wells (H9, H10, H11 and H12). A sterile reagent reservoir was used to hold the bacterial-complement suspension. Where multiple plates were being run, the bacteria-complement suspension was mixed with a pipette, between additions to successive plates.
3. 12.5 μl bacteria diluted at the final dilution was added to the “serum and bacteria only” control wells.
   E. Serum Bactericidal Reaction

Plates were covered with their lids and the microtiters plates were incubated on a horizontal rotator shaker at 200 rpm at 37±2° C., No CO2 for 60±5 min. This step allowed the killing of bacteria by sera and complement.

F. Viable Bacteria Enumeration

1. Sterile water (RCM-66) was added to the Millipore™ 96 well HV plate, 100 μl per well.
2. Filtration: The HV plate was placed on the Millipore™ MultiScreen™ filtration system and the pump was turned on. Note: Make sure meter is showing 10±2 in. Hg-vac. The plate contents were filtered through the membrane by pushing down the plate with the lid to the base of the unit until the plate was fully emptied and no liquid remained visible.
3. TSB broth (room temperature) was added to the Millipore™ 96 well HV plate; 100 μl per well.
4. The contents in the wells from SBA reaction were carefully mixed (4-6 times) and the desired volume of sample (e.g. 5 μl) from each well was transferred into the corresponding wells of the HV plates using a multichannel pipette.
   Note: The same volume (either 5 μL) must be added to all wells in the HV plates.
   Note: The transferred volume (5 μL) is usually 1/10 of the total volume in each well, except that for the “specificity control” wells, only 5 μL is transferred, but the total volume for those wells is 60 μL. This disproportion would only affects “serum and bacteria only” wells, won't make any difference on the results of test wells.
5. Filtration: Step 2 was repeated.
   Note: Filtration can happen very fast (within 5-10 seconds). Therefore, monitor the filtration carefully to prevent over-drying of the plate. Note that too much broth remaining in the wells will give high background and poor quality staining. Remove plate from the manifold immediately after most of the broth is drawn through the wells, but avoiding over-drying and over-filtration. Carefully monitor wells during filtration to observe that well contents have been vacuum-filtered, as discernable by the disappearance of liquid from the outside edges of the wells. Remove plate immediately after this occurs.
6. After filtration, the base of the HV plate unit was pat dry on a paper towel. The plate was wrapped with plastic film.
7. The wrapped plate was inserted in a plastic bag (to keep moisture inside of plate) and incubated overnight at 37±2° C., 5±1% CO₂, for a period of 15-17 hours (avoiding the overgrowth of colonies). The plates were placed in an upright position in the incubator.

Volume in Each Well at this Point:

| | Positive | Specificity | C′ control | Serum bacteria | c Medium |
|---|---|---|---|---|---|
| | serum | control | (bacteria + C′) | (serum control) | control |
| D-PBS-Glc buffer | — | — | 25 μl | 12.5 μl | 50 μl |
| Polysaccharide | — | 10 μl | — | — | — |
| Diluted serum | 25 μl | 25 μl | — | 25 μl | — |
| C′ | 12.5 μl | 12.5 μl | 12.5 μl | — | — |
| Bacteria | 12.5 μl | 12.5 μl | 12.5 μl | 12.5 μl | — |

G. Colony Staining and Counting
1. Only fresh-filtered stain was used for the HV plates. 0.01% Coomassie blue was filtered with Whatman No. 5 filter paper to remove indissoluble precipitates in the stain. (approximately 5 ml of stain required for each HV plate).
2. Colony staining: The HV plate was placed on the Millipore™ filtration system without pushing down. (Note: the plate is off the vacuum). The pump was turned on. Note: Make sure meter is showing 10±2 in. Hg-vac. 50 μl per well of filtered Coomassie blue was added to the plate using the Matrix Multichannel pipette. The plate was only stained for as long as the Coomassie blue was added to whole plate. Then, the plate was pushed down onto the filtration manifold to empty the contents by vacuum filtration.
3. Destaining: Once the plate was fully emptied, 100 μl per well Coomassie blue diluent was immediately added to each well and the plate was kept under the vacuum until no liquid was visible. This step removed most non-specific staining.
4. The base of the plate was pat dry on a paper towel or equivalent and the base was separated from the 96 well multiscreen plate.
5. The plate was dried upside down in a Biosafety hood with circulating air, until wells were completely dried (approximately 10± minutes).

6. ImunoSpot™ counter setting for colony counting

| | Standard setting | |
|---|---|---|
| | (medium size | |
| | colonies) | (small colonies) |
| Sensitivity: | 100 | 140 |
| Spot size: | 0.12 | 0.06 |
| Separation tolerance: | 10.0 | 10.0 |
| Diffuse counting is: | on | on |
| Diffuseness: | 20 | 20 |
| Overdeveloped area handling is: | off | off |
| Color system: | Blue | Blue |
| Detailed counting is: | off | off |
| Background balance is: | on | on |
| Background balance: | 110 | 40 |
| Hole filling is: | on | off |

Note: ImmunoSpot™ settings should be calibrated and adjusted (at 3 month intervals) by comparison with counts obtained by human readers. The longer the overnight incubation, the bigger the spot size.
7. The plate was scanned in the imaging system, the image was printed and the data was counted. The image data were saved as tiff files on CD-R disk.
   H. SBA Titer Calculations

The bactericidal titer is the reciprocal of the serum dilution with at least 50% killing, when compared to the average growth in the complement control wells. Occasionally, sera with high titers need to be re-tested at higher initial dilutions than 1:4 (for example, starting from 1:64) to determine the phagocytic titer. Serum samples with phagocytic titers <4 are reported as a titer of 2 for purposes of data analysis.

### I. Experimental Results

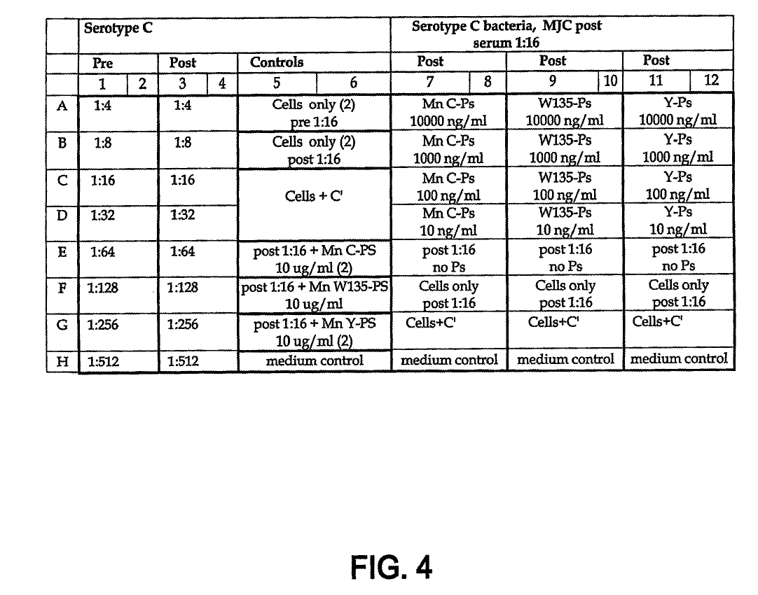

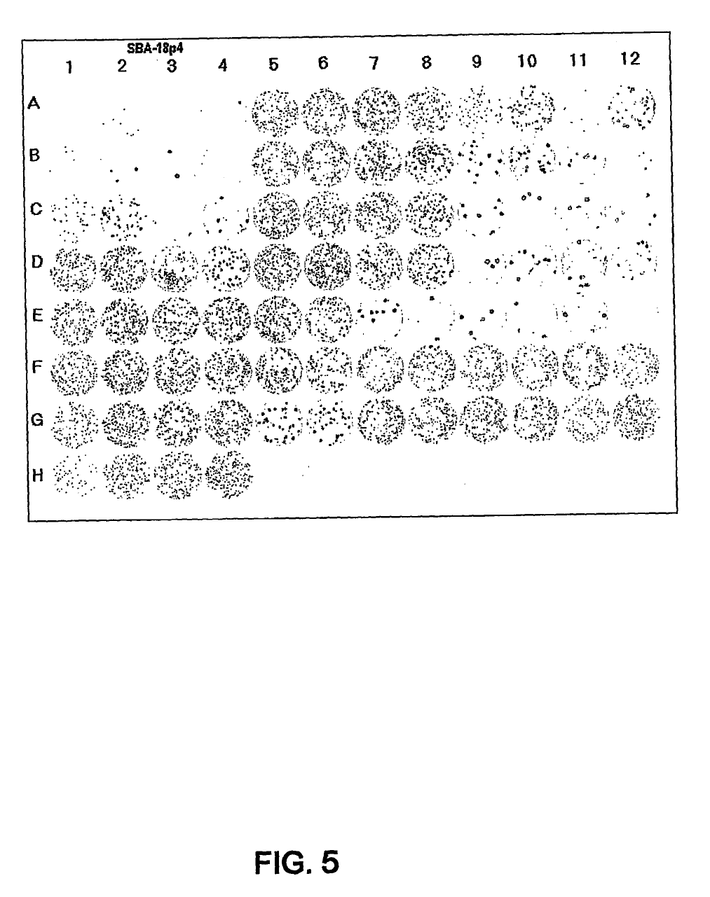

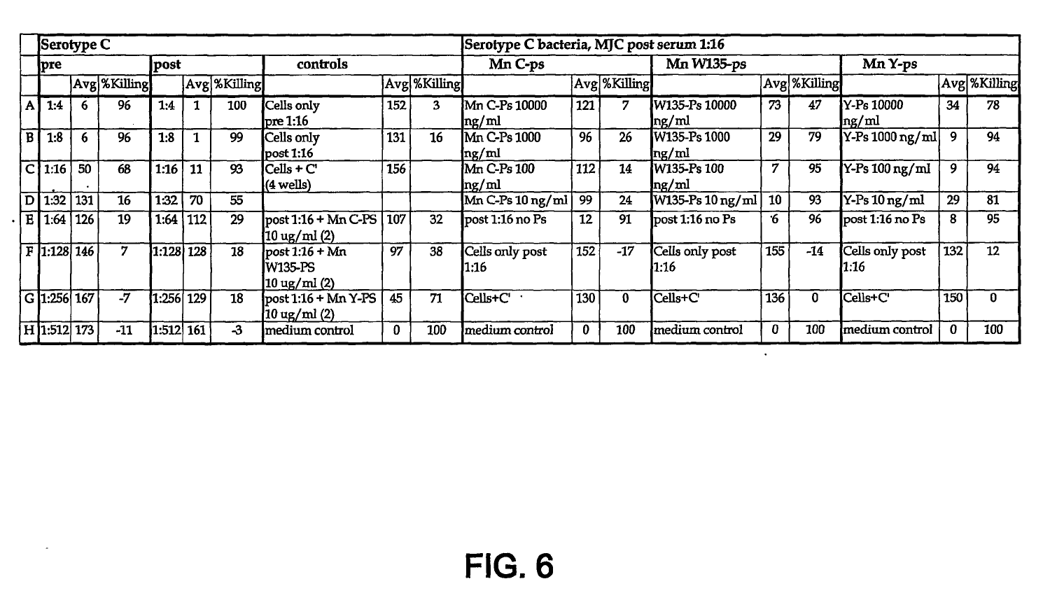

SBA assays were individually run with bacterial samples of N. meningitidis serotypes A, B, C, Y, and W135, and E. coli. Bacterial growth or lack thereof (due to the effect of an antibiotic substance) was able to be detected and analyzed within the multi-well plates. FIG. 4 shows the basic assay template set-up utilized to run the experiment with serum samples of N. meningitidis serotype C. FIGS. 5 and 6, respectively, show the colony read-out obtained from the wells both pictorially and numerically.

### Example 5

### Procedure for Validation of Opsonophagocytic Assay

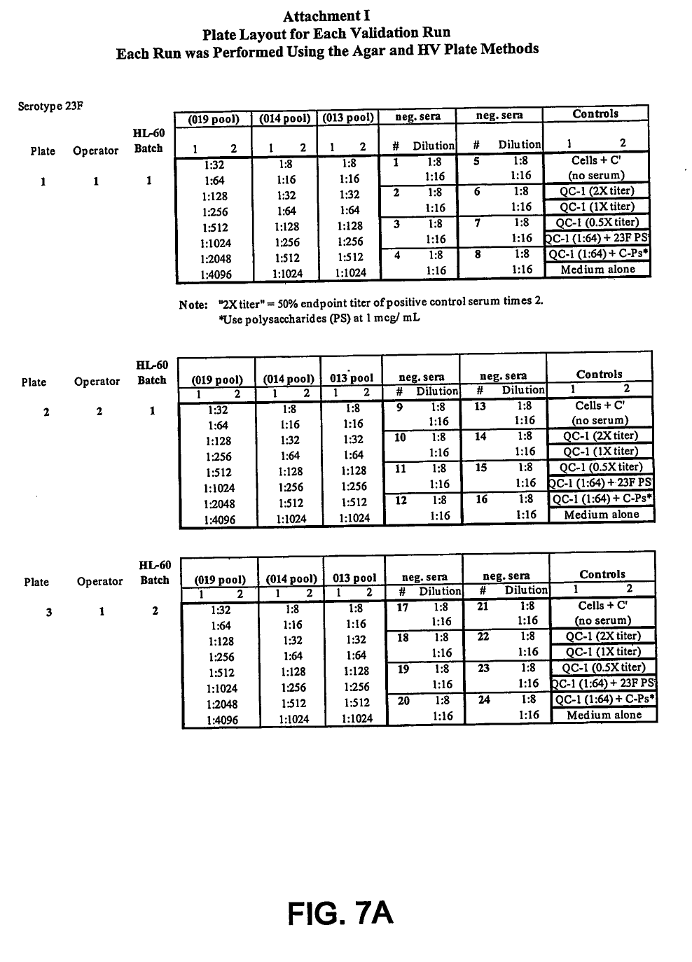

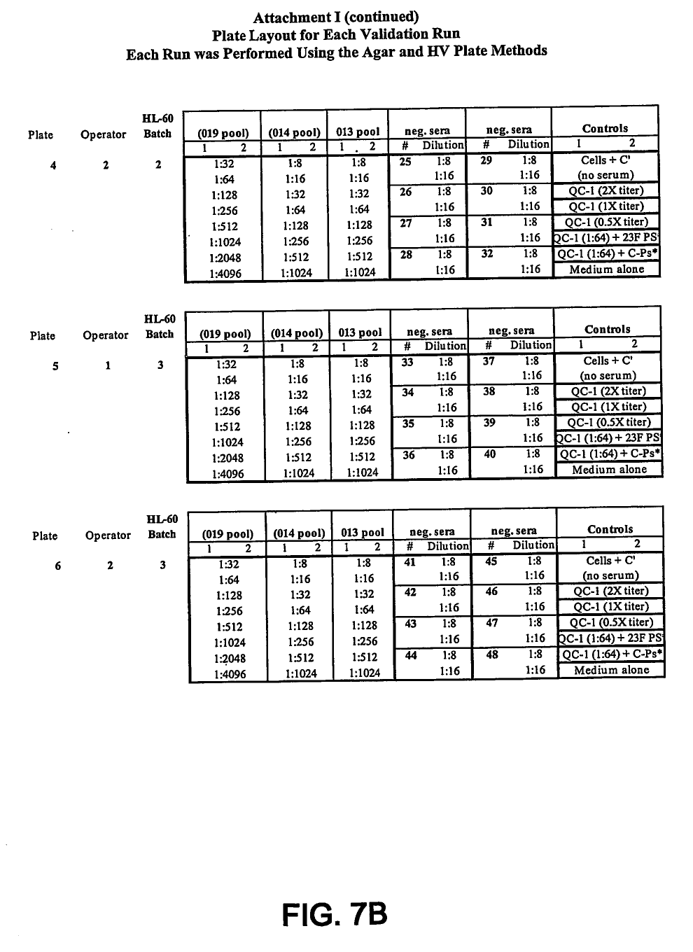

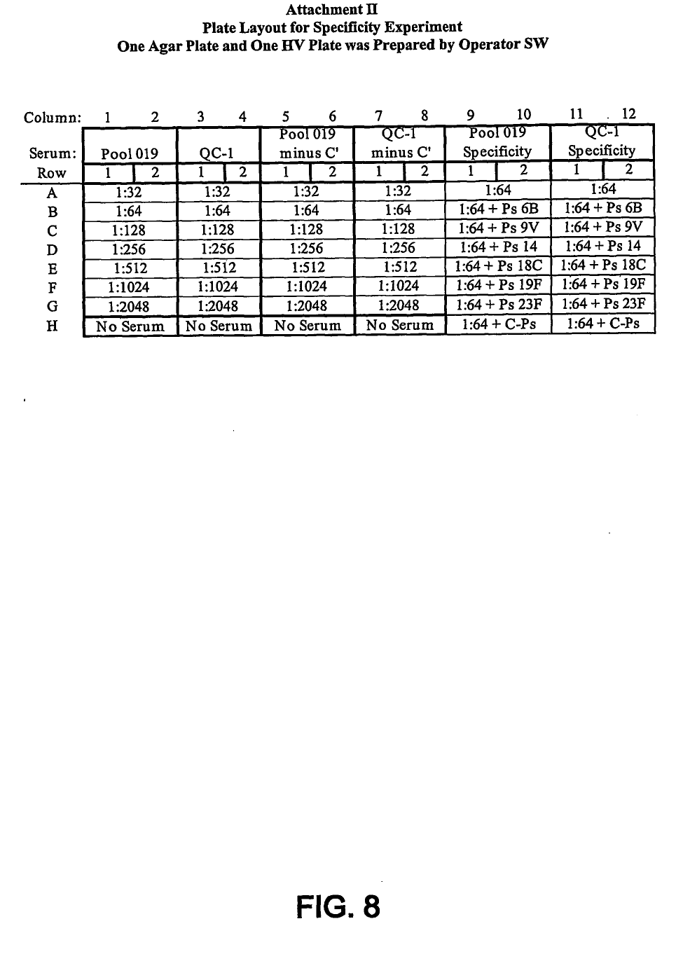

Analytical validation of the OPK assay for Streptococcus pneumoniae serotype 23F was performed in six assay runs utilizing two different operators (LS and SW), and three cell batches (p38, p39, and p71). Each of the six runs was performed using two types of plates (agar and HV). The agar plates were manually counted whereas the HV plates were counted both manually and via automation under “standard” and “high” sensitivity. Test samples included in the validation consisted of 48 ELISA negative samples, and three pools of pediatric sera ranging from low/negative to high OPK response as assessed in preliminary runs. The serum pools were tested within each run while the negative samples were evenly divided across runs. Each plate also included (1) four “No Serum” control wells containing bacteria, complement, and HL-60 cells but no antisera; (2) two “Medium Only” control wells, (3) a positive control sera (QC-1) tested at three dilutions, and (4) two specificity controls (QC-1 serum tested with 23F PS and QC-1 serum tested with C-PS). The experimental layout is provided in FIG. 7. Specificity was also assessed by determining the ability of polysaccharides of a known serotype (6B, 9V, 14, 18C, 19F, 23F, and C-Ps at 1 μg/ml) to inhibit killing by positive control antisera (Pool 019 and QC-1 tested at the 1:64 dilution). The experimental layout for assessing specificity is provided in FIG. 8. OPK titers were determined by running serial 2-fold dilution of serum in duplicate assay wells.

The Results of the Analytical Validation are Summarized Below:

| Assay Characteristic | Validation Result |
|---|---|
| Count Variability | The automated counting method (standard sensitivity) was more |
| | consistent than the manual method. Within the HV plate, the |
| | inter-run and intra-run % RSD's were 1.7% and 2.9%, |
| | respectively, for automated counting as compared to 14.3% and |
| | 14.2%, respectively, for manual counting. |
| Relationship Between | The relationship between colony count and volume is linear |
| Count Level and | between 5 μl and 15 μl. For every 2-fold increase in volume, the |
| Volume Plated | number of colonies is increased by 1.59-fold (95% CI = (1.33, |
| | 1.89)). |
| CFU Level | Relative to CFU levels in the central wells, CFU levels in the |
| Comparison: Periphery | periphery wells were reduced by 3.1% on average (95% CI = (0.2, |
| Wells versus Central | 9 CFU)). The percent reduction in CFU levels tended to increase |
| Wells | with increasing plate volume as the reductions at 5 ul, 10 ul, and |
| | 15 ul were 1.1%, 3.3%, and 4.3%, respectively. |
| Extra-variability | Extra-variability is assessed on the square roots of the replicate |
| | counts. The specification limit, applied to the range of the |
| | transformed counts, is 2.57. |
| Ruggedness | Differences in OPK titer between cell passages (p38-p71), |
| (Cell Passage, Operator, | operators, plates and counting methods were all within 2-fold on |
| Plate, Counting | average, and therefore the assay is judged to be acceptably rugged |
| Method) | to changes in these parameters. There was no evidence that the |
| | counting method within the HV plate (Manual, Standard, and |
| | High) affected titer. While the HV and Agar plates resulted in |
| | similar titer levels on average, individual results tended to be |
| | more variable on the Agar plate than on the HV plate. |
| Precision | Variability for the HV plates did not exceed that for the Agar |
| | plates. With respect to reported titers, there was no evidence of a |
| | difference in assay precision between the manual and automated |
| | counting methods within the HV plate. The assay precision |
| | (percent relative standard deviation in OPK titer) for the HV |
| | plates is approximately 70% which is acceptable for an endpoint |
| | dilution assay. |
| Sero-Status Cutoff | The titer distribution of the negative control samples |
| | conservatively supports a serostatus cutoff of 1:8 in the OPK |
| | assay. Thus, samples having a titer of <1:8 are considered |
| | negative in the assay. |
| Specificity | Of the seven polysaccharides evaluated (6B, 9V, 14, 18C, 19F, |
| | 23F, and C-Ps at 1 μg/ml), only 23F inhibited killing. |
| Assay Controls | The following six requirements are recommended in order to |
| | qualify an assay run: (1) the positive control must be positive |
| | (i.e., >50% inhibition) at least at one of three dilutions tested, |
| | 1:0.5X, 1:X, and 1:2X, where X represents the endpoint titer for |
| | the positive control; (2) The positive control tested at a dilution of |
| | 1:0.5X in the presence of 23F-Ps at 1 μg/ml must be negative |
| | (i.e., ≦50% inhibition); (3) The positive control tested at a |
| | dilution of 1:0.5X in the presence of C-Ps at 1 μg/ml must be |
| | positive (i.e., >50% inhibition); (4) The replicate counts within |
| | the “Medium Only” and “No Serum” control wells must satisfy |
| | the extra-variability criteria; (5) The average number of colonies |
| | across the four “No Serum” control wells must be ≧25 after |
| | correcting for the “Medium Only” control wells and <200 (<300) |
| | for Agar (HV) plates prior to correcting for the “Medium Only” |
| | control wells; and (6) The average number of colonies in the two |
| | “Medium Only” control wells must be ≦25% of the average |
| | number of CFU in the four “No Serum” control wells. Any |
| | exceptions would require appropriate notation in order to accept |
| | the run. |

### Example 6

### SBA Assay Application: Rapid Selection of Functional Monoclonal Antibodies that Kill Neisseria Meningitidis Serotype B

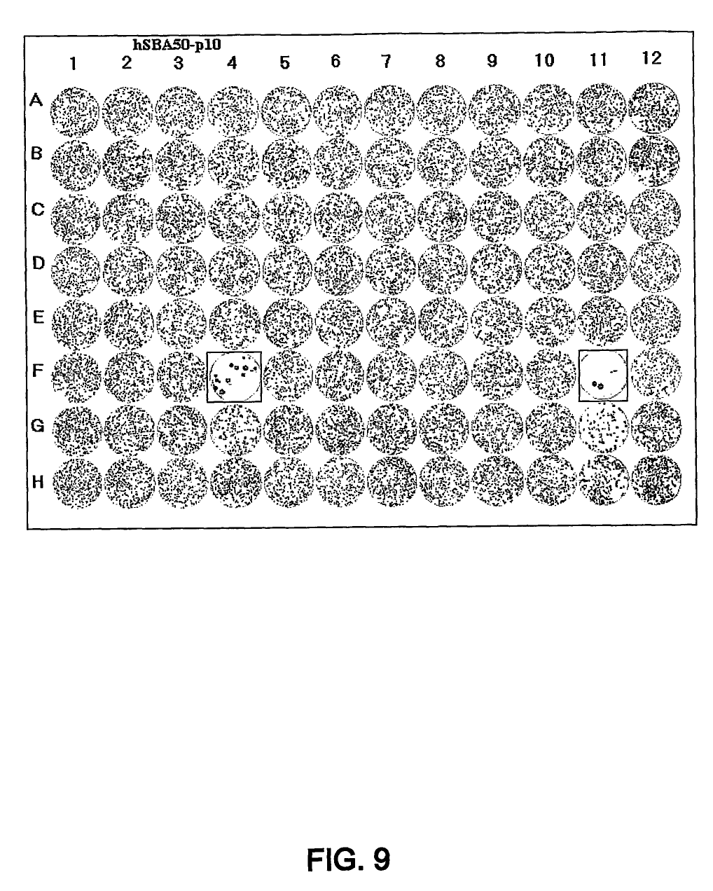

The selection of hybridomas that produce monoclonal antibodies typically involves the screening of hundreds of culture supernatants for antibodies of the desired specificity and function. Usually, this involves a two-step selection process in which hybridomas are screened for the ability to produce antibodies that bind to an antigen in an enzyme-based immunoassay (EIA). Subsequently, the supernatants are screened for functional activity (e.g. utilizing an OPK or SBA assay). Utilizing the methods described in the instant application, these separate steps are combined into a single rapid screening assay. Results employing such an assay for the screening of hybridoma culture supernatants for serum bactericidal activity against N. meningitidis serotype B are exemplified in FIG. 9. In the Figure, one can clearly see serum bactericidal activity in the hybridoma culture supernatants of wells F4 and F11 which, incidentally, have only a few bacterial colonies as opposed to the remaining wells that all have a large numbers of colonies. It is important to note that antibiotics must not be added to hybridoma culture medium as they will interfere with the rapid SBA screening procedure by killing the test bacteria.

What is claimed is:

1. A method for enumerating microbial colonies in a sample comprising:
(a) transferring a sample comprising a microbe(s) of interest in a liquid medium to the wells of a multi-well filter plate;
(b) removing excess media from the wells;
(c) allowing sufficient time for the microbe(s) to grow into discrete colonies on residual growth media captured within and under the filter plate; and
(d) enumerating the microbial colonies in the sample by using a means suitable for enumeration of samples in multi-well format.

2. A method in accordance with claim 1 wherein the microbe(s) are bacteria and the microbial colonies are bacterial colonies.

3. A method in accordance with claim 1 wherein the microbe(s) are yeast and the microbial colonies are colonies of yeast.

4. A method in accordance with claim 1 wherein the microbe(s) are fungi and the microbial colonies are fungal colonies.

5. A method in accordance with claim 1 wherein the multi-well filter plate of step (a) comprises growth medium.

6. A method in accordance with claim 1 wherein the multi-well filter plate is a 96 well filter plate.

7. A method in accordance with claim 2 wherein the bacteria are grown on the filter plate for a period of 14-18 hours.

8. A method in accordance with claim 1 wherein the filter plate is a Millipore™ 96 well HV plate.

9. A method in accordance with claim 1 wherein the filter plate is a Millipore™ Multiscreen™ HV 0.45 μm Opaque Sterile Filtration plate.

10. A method in accordance with claim 1 wherein the excess media is removed by vacuum filtration.

11. A method in accordance with claim 1 wherein the excess media is removed by centrifugation.

12. A method in accordance with claim 1 wherein the enumeration of microbial colonies is accomplished with a device capable of acquiring images and/or information from wells in multi-well format.

13. A method in accordance with claim 12 wherein the device is capable of acquiring images and/or information from wells in 96 well format.

14. A method in accordance with claim 12 wherein the number of bacteria in the sample is determined using a computer-assisted video imaging and analysis system.

15. A method in accordance with claim 12 wherein the number of bacteria in the sample is determined using an ImmunoSpot™ Analyzer.

16. A method in accordance with claim 1 wherein the microbe(s) prior to step (a) had been contacted with an antimicrobial agent.

17. A method in accordance with claim 16 wherein the microbe(s) prior to step (a) had been contacted with an antimicrobial antiserum.

18. A method in accordance with claim 16 wherein the microbe(s) prior to step (a) were further contacted with complement or active components thereof.

19. A method in accordance with claim 17 wherein the microbe(s) prior to step (a) were further contacted with an effector cell capable of engulfing the microbe(s).

20. A method in accordance with claim 18 wherein the effector cell is a phagocyte.

21. A method in accordance with claim 18 wherein the effector cell is a differentiated HL-60 cell.

22. A method in accordance with claim 18 wherein the effector cell is a peripheral blood polymorphonuclear leukocyte.

23. A method in accordance with claim 2 wherein the bacteria is a gram-positive bacteria.

24. A method in accordance with claim 2 wherein the bacteria is a gram-negative bacteria.

25. A method in accordance with claim 2 wherein the bacteria is a pathogenic microorganism.

26. A method in accordance with claim 2 wherein the bacteria is Streptococcus pneumoniae.

27. A method in accordance with claim 2 wherein the bacteria is Neisseria meningitidis.

28. A method in accordance with claim 2 wherein the bacteria is Escherichia coli.

29. A method in accordance with claim 2 wherein the bacteria is Staphylococcus aureus.

30. A method in accordance with claim 2 wherein the bacteria is Bacillus anthracis.

31. A method for analyzing microbe(s), their growth and/or viability in a sample comprising:
(a) transferring a sample comprising a microbe(s) of interest in a liquid medium to the wells of a multi-well filter plate;
(b) removing excess media from the wells;
(c) allowing sufficient time for the microbe(s) to grow into discrete colonies on residual growth media captured within and under the filter plate; and
(d) analyzing the microbe(s), their growth and/or viability in the sample by a means suitable for analysis of samples in multi-well format.

32. A method in accordance with claim 30 wherein the microbe(s) are bacteria.

33. A method in accordance with claim 30 wherein the microbe(s) are yeast.

34. A method in accordance with claim 30 wherein the microbe(s) are fungi.

35. A method in accordance with claim 30 wherein the multi-well filter plate of step (a) comprises growth medium.

36. A method in accordance with claim 33 wherein the multi-well filter plate is a 96 well filter plate.

37. A method in accordance with claim 31 wherein the bacteria are grown for a period of 14-18 hours.

38. A method in accordance with claim 30 wherein the filter plate is a Millipore™ 96 well HV plate.

39. A method in accordance with claim 30 wherein the filter plate is a Millipore™ Multiscreen™ HV 0.45 μm Opaque Sterile Filtration plate.

40. A method in accordance with claim 30 wherein the excess media is removed by vacuum filtration.

41. A method in accordance with claim 30 wherein the excess media is removed by centrifugation.

42. A method in accordance with claim 30 wherein microbe(s), their growth and/or viability is analyzed with a device capable of acquiring images and/or information from wells in multi-well format.

43. A method in accordance with claim 40 wherein the device is capable of acquiring images and/or information from wells in 96 well format.

44. A method in accordance with claim 41 wherein the number of bacteria in the sample is determined using a computer-assisted video imaging and analysis system.

45. A method in accordance with claim 41 wherein the microbe(s) are analyzed using an ImmunoSpot™ Analyzer.

46. A method in accordance with claim 30 wherein the microbe(s) prior to step (a) had been contacted with an antimicrobial agent.

47. A method in accordance with claim 30 wherein the microbe(s) prior to step (a) had been contacted with antimicrobial antiserum.

48. A method in accordance with claim 44 wherein the microbe(s) prior to step (a) were further contacted with complement or active components thereof.

49. A method in accordance with claim 45 wherein the microbe(s) prior to step (a) were further contacted with an effector cell capable of engulfing the microbe(s).

50. A method in accordance with claim 46 wherein the effector cell is a phagocyte.

51. A method in accordance with claim 46 wherein the effector cell is a differentiated HL-60 cell.

52. A method in accordance with claim 46 wherein the effector cell is a peripheral blood polymorphonuclear leukocyte.

53. A method in accordance with claim 31 wherein the bacteria is a gram-positive bacteria.

54. A method in accordance with claim 31 wherein the bacteria is a gram-negative bacteria.

55. A method in accordance with claim 31 wherein the bacteria is a pathogenic microorganism.

56. A method in accordance with claim 31 wherein the bacteria is Streptococcus pneumoniae.

57. A method in accordance with claim 31 wherein the bacteria is Neisseria meningitidis.

58. A method in accordance with claim 31 wherein the bacteria is Escherichia coli.

59. A method in accordance with claim 31 wherein the bacteria is Staphylococcus aureus.

60. A method in accordance with claim 31 wherein the bacteria is Bacillus anthracis.

61. A method for evaluating antimicrobial agents comprising:
(a) contacting a sample comprising a microbe(s) of interest with the antimicrobial agent;
(b) transferring the sample comprising the microbe(s) of interest in a liquid medium to the wells of a multi-well filter plate;
(c) removing excess media from the wells;
(d) allowing sufficient time for the microbe(s) to grow into discrete colonies on residual growth media captured within and under the filter plate; and
(e) evaluating the effect of the antimicrobial agent on the growth and/or viability of the microbe(s) with a means suitable for analysis of samples in multi-well format.

62. A method for evaluating antimicrobial agents comprising:
(a) transferring a sample comprising a microbe(s) of interest in a liquid medium to the wells of a multi-well filter plate;
(b) contacting the sample comprising the microbe(s) of interest with the antimicrobial agent;
(c) removing excess media from the wells;
(d) allowing sufficient time for the microbe(s) to grow into discrete colonies on residual growth media captured within and under the filter plate; and
(e) evaluating the effect of the antimicrobial agent on the growth and/or viability of the microbe(s) with a means suitable for analysis of samples in multi-well format.

63. A method in accordance with claim 59 wherein the antimicrobial agent is a monoclonal antibodies present within hybridoma culture supernatant.
